(19) United States
(12) Patent Application Publication
(10) Pub. No.: US 2021/0071259 A1
(43) Pub. Date: Mar. 11, 2021

(54) METHOD FOR ASSISTING DETECTION OF HEAD AND NECK CANCER

(71) Applicant: HIROSHIMA UNIVERSITY, Higashihiroshima-shi, Hiroshima (JP)

(72) Inventors: Hidetoshi TAHARA, Hiroshima-shi (JP); Makoto TAHARA, Tokyo (JP)

(73) Assignee: HIROSHIMA UNIVERSITY, Higashihiroshima-shi, Hiroshima (JP)

(21) Appl. No.: 16/771,983

(22) PCT Filed: Dec. 13, 2018
(86) PCT No.: PCT/JP2018/045994

§ 371 Date: Jun. 11, 2020

(30) Foreign Application Priority Data
Dec. 13, 2017 (JP) 2017-238856

(51) Int. Cl. C12Q 1/6886

(52) U.S. Cl. CPC C12Q 1/6886; C12Q 2600/178

(57) ABSTRACT

The present invention aims at providing a method of assisting the detection of head and neck cancer with high accuracy. The present invention provides a method of assisting the detection of head and neck cancer, which includes using as an index the abundance of at least one of miRNAs, isoform miRNAs (isomiRs), precursor miRNAs, transfer RNA fragments (tRFs), or non-coding RNA fragments (LincRNAs or MiscRNAs) contained in a test sample isolated from a living body. whose nucleotide sequence is represented by any one of SEQ ID NOs: 162, 160, 145, 143, 146, 140, 141, 1 to 139, 142, 144, 147 to 159, and 161, wherein a higher abundance of at least one of the miRNAs, isomiRs, precursor miRNAs, transfer RNA fragments, or non-coding RNA fragments whose nucleotide sequence is represented by any one of SEQ ID NOs: 1 to 71 and 118 to 136 than that of healthy subjects or a lower abundance of at least one of the miRNAs, isomiRs, precursor miRNAs, transfer RNA fragments, or non-coding RNA fragments whose nucleotide sequence is represented by any one of SEQ ID NOs: 72 to 117 and 137 to 162 than that of healthy subjects indicates a higher likelihood of having head and neck cancer.

### TECHNICAL FIELD

The present invention relates to a method of assisting the detection of head and neck cancer.

### BACKGROUND ART

Head and neck cancer refers to cancer that occurs in a body region below the brain and above the clavicles. Vital functions such as breathing and eating, and socially important daily life functions such as speaking, tasting, and hearing are predominantly related to the head and neck region. Thus, a therapy to treat cancer while keeping the balance between curability and QOL is needed because any lesion in the head and neck region may directly affect QOL. Additionally, aesthetic considerations are also necessary because the head and neck region is involved in maintenance of facial morphology and/or in expression of feelings.

As methods to detect such cancer including head and neck cancer, methods in which the abundance of microRNA (hereinafter referred to as “miRNA”) in blood is used as an index are proposed (Patent Documents 1 to 5).

### PRIOR ART DOCUMENTS

### Patent Documents

Patent Document 1 WO 2009/133915
Patent Document 2 WO 2012/161124
Patent Document 3 JP 2013-539018 T
Patent Document 4 JP 2015-502176 T
Patent Document 5 JP 2015-51011 A

### SUMMARY OF THE INVENTION

### Problem to be Solved by the Invention

As described above, various miRNAs have been proposed as indexes for the detection of cancer including head and neck cancer and, needless to say, it is advantageous if head and neck cancer can be detected with higher accuracy.

Thus, an object of the present invention is to provide a method of assisting the detection of head and neck cancer which assists in highly accurate detection of head and neck cancer.

### Means for Solving the Problem

As a result of intensive study, the inventors newly found miRNAs, isoform miRNAs (isomiRs), precursor miRNAs. transfer RNA fragments (tRFs), and non-coding RNA fragments (LincRNAs, MiscRNAs) which increase or decrease in abundance in head and neck cancer. and discovered that use of those RNA molecules as indexes enables highly accurate detection of head and neck cancer, and thereby completed the present invention.

That is, the present invention provides the followings.
(1) A method of assisting the detection of head and neck cancer, using as an index the abundance of at least one of miRNAs, isoform miRNAs (isomiRs), precursor miRNAs, transfer RNA fragments (tRFs), or non-coding RNA fragments (LincRNAs or MiscRNAs) contained in a test sample isolated from a living body, whose nucleotide sequence is represented by any one of SEQ ID NOs: 162, 160, 145, 143, 146, 140, 141, 1 to 139, 142, 144, 147 to 159, and 161, wherein a higher abundance of at least one of the miRNAs, isomiRs, precursor miRNAs, transfer RNA fragments, or non-coding RNA fragments whose nucleotide sequence is represented by any one of SEQ ID NOs: 1 to 71 and 118 to 136 than that of healthy subjects or a lower abundance of at least one of the miRNAs, isomiRs, precursor miRNAs, transfer RNA fragments, or non-coding RNA fragments whose nucleotide sequence is represented by any one of SEQ ID NOs: 72 to 117 and 137 to 162 than that of healthy subjects indicates a higher likelihood of having head and neck cancer.
(2) The method according to (1), wherein the abundance of at least one of miRNAs, isoform miRNAs (isomiRs), precursor miRNAs, transfer RNA fragments (tRFs), or non-coding RNA fragments (LincRNAs or MiscRNAs) whose nucleotide sequence is represented by any one of SEQ ID NOs: 162, 160, 145, 143, 146, 140, and 141 is used as an index.
(3) The method according to (1), wherein the abundance of at least one of miRNAs, isoform miRNAs (isomiRs), precursor miRNAs, or transfer RNA fragments whose nucleotide sequence is represented by any one of SEQ ID NOs: 92, 2, 74, 73, 75, 84, 32, 77, 18, 1, 3 to 31, 33 to 72, 76, 78 to 83, 85 to 91, and 93 to 116 is used as an index.
(4) The method according to (3), wherein the abundance of an isomiR whose nucleotide sequence is represented by SEQ ID NO: 115 or 116 is used as an index.
(5) The method according to (3) or (4), wherein the abundance of a miRNA whose nucleotide sequence is represented by SEQ ID NO: 11 and a miRNA whose nucleotide sequence is represented by SEQ ID NO: 30 is used as an index.
(6) The method according to (3) or (4), wherein the abundance of a miRNA whose nucleotide sequence is represented by SEQ ID NO: 11 and a miRNA whose nucleotide sequence is represented by SEQ ID NO: 26 is used as an index.
(7) The method according to (3) or (4), wherein the abundance of a miRNA whose nucleotide sequence is represented by SEQ ID NO: 11 and a miRNA whose nucleotide sequence is represented by SEQ ID NO: 117 is used as an index.
(8) The method according to (3) or (4), wherein the abundance of a miRNA whose nucleotide sequence is represented by SEQ ID NO: 30 and a miRNA whose nucleotide sequence is represented by SEQ ID NO: 118 is used as an index.
(9) The method according to (1), wherein the abundance of a miRNA whose nucleotide sequence is represented by SEQ ID NO: 157 and a miRNA whose nucleotide sequence is represented by SEQ ID NO: 162 is used as an index.
(10) The method according to any one of (3) to (8), wherein the head and neck cancer is tongue cancer.

### Effect of the Invention

By the method of the present invention, head and neck cancer can be highly accurately and yet conveniently detected. Thus, the method of the present invention will greatly contribute to the detection of head and neck cancer.

### MODE FOR CARRYING OUT THE INVENTION

As described above, the abundance of a specified miRNAs, isomiRs, precursor miRNAs, transfer RNA fragments (tRFs), or non-coding RNA fragments (LincRNAs or MiscRNA) (hereinafter sometimes referred to as “miRNAs or the like” for convenience) contained in a test sample isolated from a living body is used as an index in the method of the present invention. The nucleotide sequence of these miRNAs or the like themselves are as shown in Sequence Listing. The list of miRNAs or the like used in the method of the present invention is presented in Tables 1-1 to 1-7 below.

TABLE 1-1

| SEQ | | | | Length | |
|---|---|---|---|---|---|
| ID | | | | (nucleo- | |
| NO: | Class | Archetype | Type | tides) | Sequence |
| 1 | tRF | tRNA-Gly-CCC-1-1// . . . *1 | Exact | 30 | gcauuggugguucagugguagaauucucgc |
| 2 | tRE | tRNA-Lys-TTT-3-1// . . . *2 | Exact | 28 | cggauagcucagucgguagagcaucaga |
| 3 | tRF | tRNA-Glu-CTC-1-1// . . . *3 | Exact | 32 | ucccugguggucuagugguuaggauucggcgc |
| 4 | tRF | tRNA-Pro-TGG-2-1 | Exact | 31 | ggcucguuggucuagggguaugauucucggu |
| 5 | tRF | tRNA-Lys-TTT-3-1// . . . *4 | Exact | 31 | gcccggauagcucagucgguagagcaucaga |
| 6 | tRF | tRNA-iMet-CAT-1-1// . . . *5 | Exact | 33 | agcagaguggcgcagcggaagcgugcugggccc |
| 7 | tRF | tRNA-Lys-CTT-1-1// . . . *6 | Exact | 31 | gcccggcuagcucagucgguagagcauggga |
| 8 | tRF | tRNA-iMet-CAT-1-1// . . . *7 | Exact | 31 | agcagaguggcgcagcggaagcgugcugggc |
| 9 | isomiR | mir-183 | Mature 5′ sub | 21 | auggcacugguagaauucacu |
| 10 | isomiR | mir-223 | Mature 3′ sub | 17 | ugucaguuugucaaaua |
| 11 | miRNA | mir-150 | Mature 5′ | 22 | ucucccaacccuuguaccagug |
| 12 | isomiR | mir-223 | Mature 3′ super | 24 | ugucaguuugucaaauaccccaag |
| 13 | tRF | tRNA-Lys-CTT-1-1// . . . *8 | Exact | 28 | cggcuagcucagucgguagagcauggga |
| 14 | isomiR | mir-150 | Mature 5′ super | 23 | ucucccaacccuuguaccagugc |
| 15 | isomiR | mir-150 | Mature 5 sub | 19 | ucucccaacccuuguacca |
| 16 | tRF | tRNA-Pro-AGG-1-1// . . . *9 | Exact | 30 | ggcucguuggucuagggguaugauucucgc |
| 17 | isomiR | mir-146b | Mature 5′ super | 23 | ugagaacugaauuccauaggcug |
| 18 | tRF | tRNA-iMet-CAT-1-1// . . . *10 | Exact | 30 | agcagaguggcgcagcggaagcgugcuggg |
| 19 | isomiR | mir-361 | Mature 3′ super | 24 | ucccccaggugugauucugauuug |
| 20 | isomiR | mir-223 | Mature 3′ sub/ | 21 | ucaguuugucaaauaccccaa |
| | | | super | | |
| 21 | precursor | mir-223 | precursor miRNA | 15 | ugucaguuugucaaa |
| 22 | precursor | mir-223 | precursor miRNA | 16 | ugucaguuugucaaau |
| 23 | isomiR | mir-146a | Mature 5′ sub | 20 | ugagaacugaauuccauggg |
| 24 | isomiR | mir-150 | Mature 5′ sub | 20 | ucucccaacccuuguaccag |
| 25 | isomiR | mir-223 | Mature 3′ sub | 18 | ugucaguuugucaaauac |
| 26 | miRNA | mir-29a | Mature 3′ | 22 | uagcaccaucugaaaucgguua |
| 27 | isomiR | mir-223 | Mature 3′ sub | 20 | ucaguuugucaaauacccca |
| 28 | miRNA | mir-339 | Mature 5′ | 23 | ucccuguccuccaggagcucacg |

TABLE 1-2

| SEQ | | | | Length | |
|---|---|---|---|---|---|
| ID | | | | (nucleo- | |
| NO: | Class | Archetype | Type | tides) | Sequence |
| 29 | isomiR | mir-223 | Mature 3′ super | 23 | ugucaguuugucaaauaccccaa |
| 30 | miRNA | mir-146b | Mature 5′ | 22 | ugagaacugaauuccauaggcu |
| 31 | isomiR | mir-365a//mir-365b | Mature 3′ sub | 21 | uaaugccccuaaaaauccuua |
| 32 | miRNA | mir-140 | Mature 5′ | 22 | cagugguuuuacccuaugguag |
| 33 | miRNA | mir-223 | Mature 3′ | 22 | ugucaguuugucaaauacccca |
| 34 | isomiR | mir-223 | Mature 3′ sub/ | 22 | gucaguuugucaaauaccccaa |
| | | | super | | |
| 35 | tRF | tRNA-Leu-AAG-1-1// . . . *11 | Exact | 16 | gguagcguggccgagc |
| 36 | isomiR | mir-150 | Mature 5′ sub | 21 | ucucccaacccuuguaccagu |
| 37 | isomiR | mir-146b | Mature 5′ super | 24 | ugagaacugaauuccauaggcugu |
| 38 | tRF | tRNA-Glu-CTC-1-1// . . . *12 | Exact | 30 | ucccugguggucuagugguuaggauucggc |
| 39 | isomiR | mir-223 | Mature 3′ sub | 20 | ugucaguuugucaaauaccc |
| 40 | isomiR | mir-145 | Mature 5′ super | 24 | guccaguuuucccaggaaucccuu |
| 41 | isomiR | mir-186 | Mature 5′ sub | 21 | caaagaauucuccuuuugggc |
| 42 | miRNA | mir-365a//mir-365b | Mature 3′ | 22 | uaaugccccuaaaaauccuuau |
| 43 | isomiR | mir-223 | Mature 3′ super | 23 | gugucaguuugucaaauacccca |
| 44 | isomiR | mir-192 | Mature 5′ sub | 20 | ugaccuaugaauugacagcc |
| 45 | tRF | tRNA-Gly-GCC-2-1// . . . *13 | Exact | 33 | gcauuggugguucagugguagaauucucgccug |
| 46 | miRNA | mir-17 | Mature 5′ | 23 | caaagugcuuacagugcagguag |
| 47 | isomiR | mir-339 | Mature 5′ sub | 19 | ucccuguccuccaggagcu |
| 48 | isomiR | mir-223 | Mature 3′ sub | 21 | ugucaguuugucaaauacccc |
| 49 | isomiR | mir-223 | Mature 3′ sub | 21 | gucaguuugucaaauacccca |
| 50 | isomiR | mir-30c-2//mir-30c-1 | Mature 5′ sub | 22 | uguaaacauccuacacucucag |
| 51 | isomiR | mir-1307 | Mature 3′ super | 23 | acucggcguggcgucggucgugg |
| 52 | miRNA | mir-29c | Mature 3′ | 22 | uagcaccauuugaaaucgguua |
| 53 | isomiR | mir-223 | Mature 3′ sub | 20 | gucaguuugucaaauacccc |
| 54 | isomiR | mir-223 | Mature 3′ super | 24 | gugucaguuugucaaauaccccaa |
| 55 | isomiR | mir-30b | Mature 5′ sub | 21 | uguaaacauccuacacucagc |
| 56 | isomiR | mir-766 | Mature 3′ sub | 21 | acuccagccccacagccucag |
| 57 | isomiR | mir-26b | Mature 3′ sub | 21 | ccuguucuccauuacuuggcu |

TABLE 1-3

| SEQ | | | | Length | |
|---|---|---|---|---|---|
| ID | | | | (nucleo- | |
| NO: | Class | Archetype | Type | tides) | Sequence |
| 58 | tRF | tRNA-Gly-CCC-1-1// . . . *14 | Exact | 22 | gcauuggugguucagugguaga |
| 59 | miRNA | let-7d | Mature 3′ | 22 | cuauacgaccugcugccuuucu |
| 60 | tRF | tRNA-Gly-CCC-1-1// . . . *15 | Exact | 25 | gcauuggugguucagugguagaauu |
| 61 | isomiR | mir-30d | Mature 5′ sub | 19 | uguaaacauccccgacugg |
| 62 | miRNA | mir-505 | Mature 3′ | 22 | cgucaacacuugcugguuuccu |
| 63 | isomiR | mir-93 | Mature 5′ sub | 22 | aaagugcuguucgugcagguag |
| 64 | isomiR | mir-30e | Mature 5′ super | 23 | uguaaacauccuugacuggaagc |
| 65 | precursor | mir-16-1//mir-16-2 | precursor miRNA | 16 | uagcagcacguaaaua |
| 66 | miRNA | mir-193a | Mature 5′ | 22 | ugggucuuugcgggcgagauga |
| 67 | isomiR | mir-320a | Mature 3′ super | 25 | aaaagcuggguugagagggcgaaaa |
| 68 | isomiR | mir-29b-1//mir-29b-2 | Mature 3′ sub | 21 | uagcaccauuugaaaucagug |
| 69 | isomiR | mir-142 | Mature 5′ sub/super | 22 | cccauaaaguagaaagcacuac |
| 70 | isomiR | mir-142 | Mature 5′ sub/super | 21 | cccauaaaguagaaagcacua |
| 71 | miRNA | mir-744 | Mature 5′ | 22 | ugcggggcuagggcuaacagca |
| 72 | isomiR | mir-200b | Mature 3′ sub | 21 | aauacugccugguaaugauga |
| 73 | isomiR | mir-181b-1//mir-181b-2 | Mature 5′ sub | 19 | uucauugcugucggugggu |
| 74 | isomiR | mir-200a | Mature 3′ sub | 18 | acugucugguaacgaugu |
| 75 | isomiR | mir-181b-1//mir-181b-2 | Mature 5′ sub | 18 | ucauugcugucggugggu |
| 76 | isomiR | mir-181b-1//mir-181b-2 | Mature 5′ sub | 20 | auucauugcugucggugggu |
| 77 | miRNA | mir-340 | Mature 3′ | 22 | uccgucucaguuacuuuauagc |
| 78 | isomiR | mir-181b-1//mir-181b-2 | Mature 5′ sub | 21 | cauucauugcugucggugggu |
| 79 | miRNA | mir-378c | Mature 3′ | 19 | acuggacuuggagucagga |
| 80 | precursor | mir-181b-1//mir-181b-2 | precursor miRNA | 17 | cauugcugucggugggu |
| 81 | isomiR | mir-145 | Mature 5′ sub | 19 | aguuuucccaggaaucccu |
| 82 | precursor | mir-181b-1//mir-181b-2 | precursor miRNA | 16 | auugcugucggugggu |
| 83 | isomiR | mir-181b-1//mir-181b-2 | Mature 5′ sub | 22 | acauucauugcugucggugggu |
| 84 | isomiR | mir-451a | Mature 5′ sub | 18 | cguuaccauuacugaguu |
| 85 | isomiR | mir-29b-1//mir-29b-2 | Mature 3′ sub | 22 | agcaccauuugaaaucaguguu |

TABLE 1-4

| SEQ | | | | Length | |
|---|---|---|---|---|---|
| ID | | | | (nucleo- | |
| NO: | Class | Archetype | Type | tides) | Sequence |
| 86 | isomiR | mir-451a | Mature 5′ sub | 17 | guuaccauuacugaguu |
| 87 | precursor | mir-181b-1//mir-181b-2 | precursor miRNA | 15 | uugcugucggugggu |
| 88 | isomiR | mir-144 | Mature 3′ sub | 17 | uacaguauagaugaugu |
| 89 | isomiR | mir-451a | Mature 5′ sub/super | 18 | guuaccauuacugaguuu |
| 90 | isomiR | mir-451a | Mature 5′ sub | 19 | accguuaccauuacugagu |
| 91 | miRNA | let-7e | Mature 5′ | 22 | ugagguaggagguuguauaguu |
| 92 | isomiR | mir-16-2 | Mature 3′ sub/super | 20 | accaauauuacugugcugcu |
| 93 | isomiR | mir-451a | Mature 5′ super | 25 | aaaccguuaccauuacugaguuuag |
| 94 | isomiR | mir-486-1 | Mature 5′ super | 23 | uccuguacugagcugccccgagg |
| 95 | isomiR | mir-126 | Mature 3′ sub | 20 | ucguaccgugaguaauaaug |
| 96 | isomiR | mir-363 | Mature 3′ sub | 19 | aauugcacgguauccaucu |
| 97 | isomiR | mir-574 | Mature 5′ sub | 21 | ugagugugugugugugagugu |
| 98 | miRNA | let-7b | Mature 5′ | 22 | ugagguaguagguugugugguu |
| 99 | miRNA | mir-144 | Mature 3′ | 20 | uacaguauagaugauguacu |
| 100 | isomiR | mir-574 | Mature 3′ sub | 21 | cacgcucaugcacacacccac |
| 101 | isomiR | let-7b | Mature 5′ sub | 21 | ugagguaguagguuguguggu |
| 102 | isomiR | mir-103a-2//mir- | Mature 3′ sub | 19 | agcagcauuguacagggcu |
| | | 103a-1//mir-107 | | | |
| 103 | isomiR | mir-126 | Mature 3′ sub | 21 | cguaccgugaguaauaaugcg |
| 104 | isomiR | mir-451a | Mature 5′ super | 24 | gaaaccguuaccauuacugaguuu |
| 105 | miRNA | mir-106b | Mature 5′ | 21 | uaaagugcugacagugcagau |
| 106 | miRNA | let-71 | Mature 5′ | 22 | ugagguaguaguuugugcuguu |
| 107 | precursor | mir-451a | precursor miRNA | 15 | uuaccauuacugagu |
| 108 | isomiR | mir-425 | Mature 5′ sub | 19 | aaugacacgaucacucccg |
| 109 | isomiR | mir-16-2 | Mature 3′ sub | 20 | ccaauauuacugugcugcuu |
| 110 | miRNA | mir-139 | Mature 5′ | 23 | ucuacagugcacgugucuccagu |
| 111 | isomiR | mir-451a | Mature 5′ super | 23 | gaaaccguuaccauuacugaguu |
| 112 | isomiR | mir-18a | Mature 5′ sub | 21 | uaaggugcaucuagugcagau |
| 113 | miRNA | mir-126 | Mature 3′ | 22 | ucguaccgugaguaauaaugcg |

TABLE 1-5

| SEQ | | | | Length | |
|---|---|---|---|---|---|
| ID | | | | (nucleo- | |
| NO: | Class | Archetype | Type | tides) | Sequence |
| 114 | isomiR | mir-550a-1//mir-550a-2//mir-550a-3 | Mature 3′ sub | 21 | ugucuuacucccucaggcaca |
| 115 | isomiR | mir-142 | Mature 3′ sub | 22 | guaguguuuccuacuuuaugga |
| 116 | isomiR | mir-142 | Mature 3′ sub | 21 | guaguguuuccuacuuuaugg |
| 117 | miRNA | mir-339 | Mature 3′ | 23 | ugagcgccucgacgacagagccg |
| 118 | miRNA | mir-17 | Mature 3′ | 22 | acugcagugaaggcacuuguag |
| 119 | MiscRNA | ENST00000363745.1// . . . *16 | Exact | 28 | cccccacugcuaaauuugacug |
| | | | | | gcuuuu |
| 120 | MiscRNA | ENST00000364600.1// . . . *17 | Exact | 31 | gcugguccgaugguaguggguua |
| | | | | | ucagaacu |
| 121 | miRNA | mir-221 | Mature 3′ | 23 | agcuacauugucugcuggguuuc |
| 122 | miRNA | mir-374b | Mature 5′ | 22 | auauaauacaaccugcuaagug |
| 123 | isomiR | mir-130a | Mature 3′ super | 23 | cagugcaauguuaaaagggcauu |
| 124 | miRNA | mir-340 | Mature 5′ | 22 | uuauaaagcaaugagacugauu |
| 125 | miRNA | mir-199a-1//mir-199a-2//mir-199b | Mature 3′ | 22 | acaguagucugcacauugguua |
| 126 | isomiR | mir-23a | Mature 3′ super | 23 | aucacauugccagggauuuccaa |
| 127 | miRNA | mir-335 | Mature 5′ | 23 | ucaagagcaauaacgaaaaaugu |
| 128 | miRNA | mir-130a | Mature 3′' | 22 | cagugcaauguuaaaagggcau |
| 129 | isomiR | mir-584 | Mature 5′ sub | 21 | uuaugguuugccugggacuga |
| 130 | MiscRNA | ENST00000363745.1// . . . *18 | Exact | 26 | cccccacugcuaaauuugacu |
| | | | | | ggcuu |
| 131 | miRNA | mir-26a-1//mir-26a-2 | Mature 5′ | 22 | uucaaguaauccaggauaggcu |
| 132 | MiscRNA | ENST00000364600.11/ . . . *17 | Exact | 32 | ggcugguccgaugguaguggguu |
| | | | | | aucagaacu |
| 133 | isomiR | mir-23a | Mature 3′ super | 22 | aucacauugccagggauuucca |
| 134 | miRNA | mir-146a | Mature 5′ | 22 | ugagaacugaauuccauggguu |
| 135 | miRNA | mir-191 | Mature 5′ | 23 | caacggaaucccaaaagcagcug |
| 136 | MiscRNA | ENST00000364600.1// . . . *17 | Exact | 31 | ggcugguccgaugguaguggguu |
| | | | | | aucagaac |
| 137 | miRNA | mir-92a-1//mir-92a-2 | Mature 3′ | 22 | uauugcacuugucccggccugu |
| 138 | isomiR | let-7b | Mature 5′ sub | 20 | ugagguaguagguugugugg |
| 139 | isomiR | mir-451a | Mature 5′ sub | 21 | aaaccguuaccauuacugagu |
| 140 | isomiR | mir-30e | Mature 5′ sub/ | 23 | guaaacauccuugacuggaagcu |
| | | | super | | |
| 141 | isomiR | let-7g | Mature 5′ sub | 21 | ugagguaguaguuuguacagu |
| 142 | miRNA | mir-486-1//mir-486-2 | Mature 5′ | 22 | uccuguacugagcugccccgag |

TABLE 1-6

| SEQ | | | | Length | |
|---|---|---|---|---|---|
| ID | | | | (nucleo- | |
| NO: | Class | Archetype | Type | tides) | Sequence |
| 143 | isomiR | mir-16-1//mir-16-2 | Mature 5′ sub | 20 | uagcagcacguaaauauugg |
| 144 | isomiR | mir-451a | Mature 5′ sub | 20 | aaaccguuaccauuacugag |
| 145 | isomiR | mir-185 | Mature 5′ sub | 21 | uggagagaaaggcaguuccug |
| 146 | isomiR | let-7a-1//let-7a-2//let-7a-3 | Mature 5′ sub | 20 | ugagguaguagguuguauag |
| 147 | isomiR | mir-92a-1//mir-92a-2 | Mature 3′ sub | 21 | uauugcacuugucccggccug |
| 148 | isomiR | mir-25 | Mature 3′ sub | 21 | cauugcacutigucucggucug |
| 149 | isomiR | mir-16-2 | Mature 3′ sub/super | 21 | accaauauuacugugcugcuu |
| 150 | isomiR | let-7f-1//let-7f-2 | Mature 5′ sub | 20 | ugagguaguagauuguauag |
| 151 | isomiR | mir-25 | Mature 3′ sub | 20 | cauugcacuugueucggucu |
| 152 | isomiR | mir-425 | Mature 5′ sub | 21 | aaugacacgaucacucccguu |
| 153 | isomiR | mir-423 | Mature 5′ sub | 21 | ugaggggcagagagcgagacu |
| 154 | isomiR | mir-484 | Mature 5′ sub | 21 | ucaggcucaguccccucccga |
| 155 | isomiR | mir-486-1//mir-486-2 | Mature 5′ sub | 21 | uccuguacugagcugccccga |
| 156 | isomiR | mir-486-1//mir-486-2 | Mature 5′ sub | 20 | uccuguacugagcugccccg |
| 157 | isomiR | let-7i | Mature 5′ sub | 21 | ugagguaguaguuugugcugu |
| 158 | isomiR | let-7d | Mature 5′ sub | 20 | agagguaguagguugcauag |
| 159 | isomiR | mir-486-1//mir-486-2 | Mature 5′ sub | 17 | uccuguacugagcugcc |
| 160 | isomiR | let-7i | Mature 5′ sub | 20 | ugagguaguaguuugugcug |
| 161 | isomiR | mir-484 | Mature 5′ sub | 20 | ucaggcucaguccccucccg |
| 162 | LincRNA | ENST00000627566.1 | Exact | 15 | ucauguaugaugcug |

*1: tRNA-Gly-CCC-1-1//tRNA-Gly-CCC-1-2//tRNA-Gly-
GCC-2-1//tRNA-Gly-GCC-2-2//tRNA-Gly-GCC-2-3//tRNA-
Gly-GCC-2-4//tRNA-Gly-GCC-2-5//tRNA-Gly-GCC-2-
6//tRNA-Gly-GCC-3-1//tRNA-Gly-GCC-5-1
*2: tRNA-Lys-TTT-3-1//tRNA-Lys-TTT-3-2//tRNA-Lys-
TTT-3-3//tRNA-Lys-TTT-3-4//tRNA-Lys-TTT-3-5//tRNA-
Lys-TTT-5-1
*3: tRNA-Glu-CTC-1-1//tRNA-Glu-CTC-1-2//tRNA-Glu-
CTC-1-3//tRNA-Glu-CTC-1-4//tRNA-Glu-CTC-1-5//tRNA-
Glu-CTC-1-6//tRNA-Glu-CTC-1-7//tRNA-Glu-CTC-2-1
*4: tRNA-Lys-TTT-3-1//tRNA-Lys-TTT-3-2//tRNA-Lys-
TTT-3-3//tRNA-Lys-TTT-3-4//tRNA-Lys-TTT-3-5//tRNA-
Lys-TTT-5-1
*5: tRNA-iMet-CAT-1-1//tRNA-iMet-CAT-1-2//tRNA-
iMet-CAT-1-3//tRNA-iMet-CAT-1-4//tRNA-iMet-CAT-1-
5//tRNA-iMet-CAT-1-6//tRNA-iMet-CAT-1-7//tRNA-
iMet-CAT-1-8//tRNA-iMet-CAT-2-1
*6: tRNA-Lys-CTT-1-1//tRNA-Lys-CTT-1-2//tRNA-Lys-
CTT-4-1
*7: tRNA-iMet-CAT-1-1//tRNA-iMet-CAT-1-2//tRNA-
iMet-CAT-1-3//tRNA-iMet-CAT-1-4//tRNA-iMet-CAT-1-
5//tRNA-iMet-CAT-1-6//tRNA-iMet-CAT-1-7//tRNA-
iMet-CAT-1-8//tRNA-iMet-CAT-2-1
*8: tRNA-Lys-CTT-1-1//tRNA-Lys-CTT-1-2//tRNA-Lys-
CTT-4-1
*9: tRNA-Pro-AGG-1-1//tRNA-Pro-AGG-2-1//tRNA-Pro-
AGG-2-2//tRNA-Pro-AGG-2-3//tRNA-Pro-AGG-2-
4//tRNA-Pro-AGG-2-5//tRNA-Pro-AGG-2-6//tRNA-Pro-
AGG-2-7//tRNA-Pro-AGG-2-8//tRNA-Pro-CGG-1-1//tRNA-
Pro-CGG-1-2//tRNA-Pro-CGG-1-3//tRNA-Pro-CGG-2-
1//tRNA-Pro-TGG-3-1//tRNA-Pro-TGG-3-2//tRNA-Pro-
TGG-3-3//tRNA-Pro-TGG-3-4//tRNA-Pro-TGG-3-5
*10: tRNA-iMet-CAT-1-1//tRNA-iMet-CAT-1-2//tRNA-
iMet-CAT-1-3//tRNA-iMet-CAT-1-4//tRNA-iMet-CAT-1-
5//tRNA-iMet-CAT-1-6//tRNA-iMet-CAT-1-7//tRNA-
iMet-CAT-1-8//tRNA-iMet-CAT-2-1
*11: tRNA-Leu-AAG-1-1//tRNA-Leu-AAG-1-2//tRNA-Leu-
AAG-1-3//tRNA-Leu-AAG-2-1//tRNA-Leu-AAG-2-2//tRNA-
Leu-AAG-2-3//tRNA-Leu-AAG-2-4//tRNA-Leu-AAG-3-
1//tRNA-Leu-TAG-1-1
*12: tRNA-Glu-CTC-1-1//tRNA-Glu-CTC-1-2//tRNA-Glu-
CTC-1-3//tRNA-Glu-CTC-1-4//tRNA-Glu-CTC-1-5//tRNA-
Glu-CTC-1-6//tRNA-Glu-CTC-1-7//tRNA-Glu-CTC-2-1
*13: tRNA-Gly-GCC-2-1//tRNA-Gly-GCC-2-2//tRNA-Gly-
GCC-2-3//tRNA-Gly-GCC-2-4//tRNA-Gly-GCC-2-5//tRNA-
Gly-GCC-2-6//tRNA-Gly-GCC-3-1//tRNA-Gly-GCC-5-1
*14: tRNA-Gly-CCC-1-1//tRNA-Gly-CCC-1-2//tRNA-Gly-
GCC-2-1//tRNA-Gly-GCC-2-2//tRNA-Gly-GCC-2-3//tRNA-
Gly-GCC-2-4//tRNA-Gly-GCC-2-5//tRNA-Gly-GCC-2-
6//tRNA-Gly-GCC-3-1//tRNA-Gly-GCC-5-1
*15: tRNA-Gly-CCC-1-1//tRNA-Gly-CCC-1-2//tRNA-Gly-
GCC-2-1//tRNA-Gly-GCC-2-2//tRNA-Gly-GCC-2-3//tRNA-
Gly-GCC-2-4//tRNA-Gly-GCC-2-5//tRNA-Gly-GCC-2-
6//tRNA-Gly-GCC-3-1//tRNA-Gly-GCC-5-1
*16: ENST00000363745.1//ENST00000516507.1
*17: ENST00000364600.1//ENST00000577883.2//
ENST00000577984.2//ENST00000516507.1//
ENST00000481041.3//ENST00000579625.2//
ENST00000365571.2//ENST00000578877.2//
ENST00000364908.1
*18: ENST00000363745.1//ENST00000364409.1//
ENST00000516507.1//ENST00000391107.1//
ENST00000459254.1

Among those miRNAs or the like, miRNAs or the like whose nucleotide sequences are represented by SEQ ID NOs: 1 to 162 (for example, “a miRNA or the like whose nucleotide sequence is represented by SEQ ID NO: 1” is hereinafter sometimes referred to simply as “a miRNA or the like represented by SEQ ID NO: 1” or “one represented by SEQ ID NO: 1” for convenience) are present in serum or exosomes.

In many of those miRNAs or the like, the logarithm of the ratio of the abundance in serum or exosomes from patients with head and neck cancer to the abundance in serum or exosomes from healthy subjects (represented by “log FC” which means the logarithm of FC (fold change) to base 2) is not less than 1.00 in absolute value, showing a statistical significance (t-test; p<0.05).

The abundance of miRNAs or the like represented by SEQ ID NOs: 1 to 71 and 118 to 136 is higher in patients with head and neck cancer than in healthy subjects, while the abundance of miRNAs or the like represented by SEQ ID NOs: 72 to 117 and 137 to 162 is lower in patients with head and neck cancer than in healthy subjects.

By a method in which among those, any of the combinations of the miRNAs represented by SEQ ID NOs: 11 and 30, SEQ ID NOs: 11 and 26, SEQ ID NOs: 11 and 117, SEQ ID NOs: 30 and 118, and SEQ ID NOs: 157 and 162 is used as an index, even early tongue cancer can be detected, as specifically described in Examples below.

The accuracy of each cancer marker is indicated using the area under the ROC curve (AUC: Area Under Curve) as an index, and cancer markers with an AUC value of 0.7 or higher are generally considered effective. AUC values of 0.90 or higher, 0.97 or higher, 0.99 or higher, and 1.00 correspond to cancer markers with high accuracy, very high accuracy, quite high accuracy, and complete accuracy (with no false-positive and false-negative events), respectively. Thus, the AUC value of each cancer marker is likewise preferably 0.90, more preferably not less than 0.97, still more preferably not less than 0.99, and most preferably 1.00 in the present invention. The ones whose nucleotide sequences are represented by SEQ ID NOs: 162, 160, 145, 143, 146, 140, and 141 are preferable due to an AUC value of 0.97 or higher; among those, ones represented by SEQ ID NOs: 162 and 160 are more preferable due to an AUC value of 0.98 or higher.

Furthermore, because the FC (fold change) in the abundance of an isomiR represented by either SEQ ID NO: 115 or SEQ ID NO: 116 is changed before and after surgery for tongue cancer, the isomiRs can be used to assess the success or failure of the surgery.

The test sample is not specifically limited, provided that the test sample is a body fluid containing miRNAs or the like; typically, it is preferable to use a blood sample (including plasma, serum, and whole blood). For the ones or the like present in serum, it is simple and preferable to use serum or plasma as a test sample. For the miRNAs or the like present in exosomes, it is preferable to use serum or plasma as a test sample, from which exosomes are isolated to extract total RNA and to measure the abundance of each miRNA or the like. The method of extracting total RNA in serum or plasma is well known and is specifically described in Examples below. The method of extracting total RNA from exosomes in serum or plasma is itself known and is specifically described in more detail in Examples below.

The abundance of each miRNA or the like is preferably measured (quantified) using a next-generation sequencer. Any instrument may be used and is not limited to a specific type of instrument, provided that the instrument determines sequences, similarly to next-generation sequencers. In the method of the present invention, as specifically described in Examples below. use of a next-generation sequencer is preferred over quantitative reverse-transcription PCR (qRT-PCR), which is widely used for quantification of miRNAs, to perform measurements from the viewpoint of accuracy because miRNAs or the like to be quantified include, for example, isomiRs, in which only one or more nucleotides are deleted from or added to the 5′ and/or 3′ ends of the original mature miRNAs thereof, and which should be distinguished from the original miRNAs when measured. Briefly, though details will be described specifically in Examples below, the quantification method can be performed as follows. When the RNA content in serum or plasma is constant, among reads measured in a next-generation sequencing analysis of the RNA content, the number of reads for each isomiR or mature miRNA per million reads is considered as the measurement value, where the total counts of reads with human-derived sequences are normalized to one million reads. When the RNA content in serum or plasma is variable in comparison with healthy subjects due to a disease, miRNAs showing little abundance variation in serum and plasma may be used. In cases where the abundance of miRNAs or the like in serum or plasma is measured, at least one miRNA selected from the group consisting of let-7g-5p, miR-425-3p, and miR-425-5p is preferably used as an internal control, which are miRNAs showing little abundance variation in serum and plasma.

The cut-off value for the abundance of each miRNA or the like for use in evaluation is preferably determined based on the presence or absence of a statistically significant difference (t-test; p<0.05, preferably p<0.01, more preferably p<0.001) from healthy subjects with regard to the abundance of the miRNA or the like. Specifically, the value of log₂ read counts (the cut-off value) can be preferably determined for each miRNA or the like, for example, at which the false-positive rate is optimal (the lowest); for example, the cut-off values (the values of log₂ read counts) for several miRNAs or the like are as indicated in Table 2. The cut-off values indicated in Table 2 are only examples, and other values may be employed as cut-off values as long as those values are appropriate to determine statistically significant difference. Additionally, the optimal cut-off values vary among different populations of patients and healthy subjects from which data is collected. However, the cut-off values indicated in Table 2 or 3 with an interval of usually ±20%, particularly ±10%, may be set as cut-off values.

Each of the above miRNAs or the like is statistically significantly different in abundance between patients with head and neck cancer and healthy subjects, and may thus be used alone as an index. However, a combination of multiple miRNAs or the like may also be used as an index, which can assist in more accurate detection of head and neck cancer.

Moreover, a method of detecting the abundance of miRNAs or the like in a test sample from human suspected of having or affected with head and neck cancer is also provided.

That is, a method of detecting the abundance of at least one of miRNAs, isoform miRNAs (isomiRs), precursor miRNAs, transfer RNA fragments (tRFs), or non-coding RNA fragments (LincRNAs or MiscRNAs) whose nucleotide sequence is represented by any one of SEQ ID NOs: 162, 160, 145, 143, 146, 140, 141, 1 to 139, 142, 144, 147 to 159, and 161 in a test sample from human suspected of having or affected with head and neck cancer is also provided, wherein the method includes the steps of:

collecting a blood sample from human; and

measuring the abundance of the miRNA(s), isoform miRNA(s) (isomiR(s)), precursor miRNA(s), transfer RNA fragment(s) (tRF(s)), or non-coding RNA fragment(s) (LincRNA(s) or MiscRNA(s)) in the blood sample by means of a next-generation sequencer or qRT-PCR,

wherein the abundance of at least one of the miRNAs, isomiRs, precursor miRNAs, transfer RNA fragments, or non-coding RNA fragments whose nucleotide sequence is represented by any one of SEQ ID NOs: 1 to 71 and 118 to 136 is higher than that in healthy subjects, or the abundance of at least one of the miRNAs, isomiRs, precursor miRNAs, transfer RNA fragments, or non-coding RNA fragments whose nucleotide sequence is represented by any one of SEQ ID NOs: 72 to 117 and 137 to 162 is lower than that in healthy subjects.

In the present invention, the term head and neck cancer includes, for example, tongue cancer (oral cavity cancer), maxillary sinus cancer, nasopharyngeal cancer, oropharyngeal cancer, hypopharyngeal cancer, laryngeal cancer, thyroid cancer, salivary gland cancer, and metastatic cervical carcinoma from unknown primary.

Additionally, in cases where the detection of head and neck cancer is successfully achieved by the above-described method of the present invention, an effective amount of an anti-head and neck cancer drug can be administered to patients in whom head and neck cancer is detected, to treat the head and neck cancer. Examples of the anti-head and neck cancer drug can include cisplatin (CDDP), 5-FU (5-fluorouracil), and docetaxel.

The present invention will be specifically described below by way of examples and comparative examples. Naturally, the present invention is not limited by the examples below.

### EXAMPLES 1 to 165

### 1. Materials and Methods

### (1) Clinical Samples

Plasma samples from 24 patients with head and neck cancer and from 10 healthy subjects were used.

### (2) Extraction of RNA in Serum

Extraction of RNA in serum was performed using the miRNeasy Mini kit (QIAGEN).
1) Each frozen plasma sample was thawed and centrifuged at 10000 rpm for 5 minutes at room temperature to precipitate aggregated proteins and blood cell components.
2) To a new 1.5-mL tube, 200 μL of the supernatant was transferred.
3) To the tube, 1000 μL of the QIAzol Lysis Reagent was added and mixed thoroughly to denature protein components.
4) To the tube, 10 μL of 0.05 nM cel-miR-39 was added as a control RNA for RNA extraction, mixed by pipetting, and then left to stand at room temperature for 5 minutes.
5) To promote separation of the aqueous and organic solvent layers, 200 μL of chloroform was added to the tube, mixed thoroughly, and left to stand at room temperature for 3 minutes.
6) The tube was centrifuged at 12000×g for 15 minutes at 4° C. and 650 μL of the upper aqueous layer was transferred to a new 2-mL tube.
7) For the separation of RNA, 975 μL of 100% ethanol was added to the tube and mixed by pipetting.
8) To a miRNeasy Mini spin column (hereinafter referred to as column), 650 μL of the mixture in the step 7 was transferred, left to stand at room temperature for 1 minute, and then centrifuged at 8000×g for 15 seconds at room temperature to allow RNA to be adsorbed on the filter of the column. The flow-through solution from the column was discarded.
9) The step 8 was repeated until the total volume of the solution of the step 7 was filtered through the column to allow all the RNA to be adsorbed on the filter.
10) To remove impurities attached on the filter, 650 μL of Buffer RWT was added to the column and centrifuged at 8000×g for 15 seconds at room temperature. The flow-through solution from the column was discarded.
11) To clean the RNA adsorbed on the filter, 500 μL of Buffer RPE was added to the column and centrifuged at 8000×g for 15 seconds at room temperature. The flow-through solution from the column was discarded.
12) To clean the RNA adsorbed on the filter, 500 μL of Buffer RPE was added to the column and centrifuged at 8000×g for 2 minutes at room temperature. The flow-through solution from the column was discarded.
13) To completely remove any solution attached on the filter, the column was placed in a new 2-mL collection tube and centrifuged at 10000×g for 1 minute at room temperature.
14) The column was placed into a 1.5-mL tube and 50 μL of RNase-free water was added thereto and left to stand at room temperature for 1 minute.
15) Centrifugation was performed at 8000×g for 1 minute at room temperature to elute the RNA adsorbed on the filter. The eluted RNA was used in the following experiment without further purification and the remaining portion of the eluted RNA was stored at −80° C.
   (3) Extraction of RNA from Exosomes

Exosomes in serum were collected as follows.

Exosome isolation was performed with the Total Exosome Isolation (from serum) from Thermo Fisher Scientific, Inc. Extraction of RNA from the collected exosomes was performed using the miRNeasy Mini kit (QIAGEN).

(4) Quantification of miRNAs or the Like

The quantification of miRNAs or the like was performed as follows.

In cases where miRNAs or the like from, for example, two groups are quantified, extracellular vesicles (including exosomes) isolated by the same method are used to purify RNAs through the same method, from which cDNA libraries are prepared and then analyzed by next-generation sequencing. The next-generation sequencing analysis is not limited by a particular instrument, provided that the instrument determines sequences.

### (5) Calculation of Cut-off Value and AUC

Specifically, the cut-off value and the AUC were calculated from measurement results as follows. The logistic regression analysis was carried out using the JMP Genomics 8 to draw the ROC curve and to calculate the AUC. Moreover, the value corresponding to a point on the ROC curve which was closest to the upper left corner of the ROC graph (sensitivity: 1.0, specificity: 1.0) was defined as the cut-off value.

### 2. Results

The results are presented in Tables 2-1 to 2-10.

TABLE 2-1

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 1 | 1 | tRF | tRNA-Gly-CCC-1-1/ . . . *1 | Exact | 30 | 1758 | 65 | 3.81 | 0.900 | 6.08 | 0.000 |
| Example 2 | 2 | tRF | tRNA-Lys-TTT-3-1// . . . *2 | Exact | 28 | 98 | 5 | 4.57 | 0.958 | 5.18 | 0.000 |
| Example 3 | 3 | tRF | tRNA-Glu-CTC-1-1// . . . *3 | Exact | 32 | 735 | 52 | 3.67 | 0.879 | 6.59 | 0.001 |
| Example 4 | 4 | tRF | tRNA-Pro-TGG-2-1 | Exact | 31 | 106 | 8 | 4.12 | 0.883 | 4.60 | 0.000 |
| Example 5 | 5 | tRF | tRNA-Lys-TTT-3-1// . . . *4 | Exact | 31 | 243 | 20 | 3.68 | 0.921 | 6.26 | 0.000 |
| Example 6 | 6 | tRF | tRNA-iMet-CAT-1-1// . . . *5 | Exact | 33 | 83 | 8 | 3.48 | 0.896 | 5.11 | 0.000 |
| Example 7 | 7 | tRF | tRNA-Lys-CTT-1-1// . . . *6 | Exact | 31 | 136 | 15 | 3.14 | 0.888 | 5.00 | 0.001 |
| Example 8 | 8 | tRF | tRNA-iMet-CAT-1-1// . . . *7 | Exact | 31 | 51 | 7 | 3.48 | 0.904 | 4.15 | 0.000 |
| Example 9 | 9 | isomiR | mir-183 | Mature 5′ sub | 21 | 91 | 12 | 2.32 | 0.777 | 5.16 | 0.007 |
| Example 10 | 10 | isomiR | mir-223 | Mature 3′ sub | 17 | 526 | 78 | 2.96 | 0.879 | 5.95 | 0.000 |
| Example 11 | 11 | miRNA | mir-150 | Mature 5′ | 22 | 17236 | 2591 | 2.39 | 0.896 | 12.74 | 0.000 |
| Example 12 | 12 | isomiR | mir-223 | Mature 3′ super | 24 | 289 | 44 | 2.59 | 0.865 | 6.70 | 0.003 |
| Example 13 | 13 | tRF | tRNA-Lys-CTT-1-l// . . . *8 | Exact | 28 | 94 | 15 | 3.10 | 0.850 | 4.72 | 0.001 |
| Example 14 | 14 | isomiR | mir-150 | Mature 5′ super | 23 | 80 | 13 | 3.10 | 0.875 | 5.51 | 0.000 |
| Example 15 | 15 | isomiR | mir-150 | Mature 5′ sub | 19 | 337 | 60 | 3.33 | 0.846 | 7.32 | 0.008 |
| Example 16 | 16 | tRF | tRNA-Pro-AGG-1-1// . . . *9 | Exact | 30 | 523 | 94 | 4.22 | 0.850 | 5.68 | 0.003 |
| Example 17 | 17 | isomiR | mir-146b | Mature 5′ super | 23 | 191 | 35 | 2.16 | 0.873 | 5.77 | 0.005 |
| Example 18 | 18 | tRF | tRNA-iMet-CAT- 1-1// . . . *10 | Exact | 30 | 125 | 22 | 3.03 | 0.931 | 5.97 | 0.000 |

TABLE 2-2

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 19 | 19 | isomiR | mir-361 | Mature 3′ super | 24 | 35 | 7 | 2.58 | 0.850 | 4.59 | 0.001 |
| Example 20 | 20 | isomiR | mir-223 | Mature 3′ | 21 | 270 | 59 | 2.56 | 0.842 | 7.16 | 0.001 |
| | | | | sub/super | | | | | | | |
| Example 21 | 21 | precursor | mir-223 | precursor | 15 | 293 | 67 | 2.14 | 0.821 | 5.68 | 0.005 |
| | | | | miRNA | | | | | | | |
| Example 22 | 22 | precursor | mir-223 | precursor | 16 | 317 | 73 | 2.71 | 0.833 | 6.67 | 0.005 |
| | | | | miRNA | | | | | | | |
| Example 23 | 23 | isomiR | mir-146a | Mature 5′ sub | 20 | 31 | 8 | 2.37 | 0.796 | 3.61 | 0.002 |
| Example 24 | 24 | isomiR | mir-150 | Mature 5′ sub | 20 | 1205 | 298 | 2.01 | 0.800 | 9.70 | 0.002 |
| Example 25 | 25 | isomiR | mir-223 | Mature 3′ sub | 18 | 356 | 92 | 2.11 | 0.838 | 6.44 | 0.009 |
| Example 26 | 26 | miRNA | mir-29a | Mature 3′ | 22 | 1384 | 355 | 2.23 | 0.858 | 9.40 | 0.000 |
| Example 27 | 27 | isomiR | mir-223 | Mature 3′ sub | 20 | 117 | 30 | 2.31 | 0.821 | 5.23 | 0.004 |
| Example 28 | 28 | miRNA | mir-339 | Mature 5′ | 23 | 39 | 10 | 2.51 | 0.796 | 3.71 | 0.002 |
| Example 29 | 29 | isomiR | mir-223 | Mature 3′ super | 23 | 110411 | 30866 | 1.80 | 0.846 | 14.64 | 0.001 |
| Example 30 | 30 | miRNA | mir-146b | Mature 5′ | 72 | 303 | 83 | 1.35 | 0.829 | 6.73 | 0.001 |
| Example 31 | 31 | isomiR | mir-365a//mir-365b | Mature 3′ sub | 21 | 55 | 16 | 1.98 | 0.833 | 4.11 | 0.003 |
| Example 32 | 32 | miRNA | mir-140 | Mature 5′ | 22 | 172 | 49 | 2.15 | 0.938 | 6.41 | 0.006 |
| Example 33 | 33 | miRNA | mir-223 | Mature 3′ | 22 | 78031 | 24601 | 1.57 | 0.825 | 15.54 | 0.002 |
| Example 34 | 34 | isomiR | mir-223 | Mature 3′ | 27 | 24932 | 7946 | 1.73 | 0.821 | 12.89 | 0.001 |
| | | | | sub/super | | | | | | | |
| Example 35 | 35 | tRF | tRNA-Leu-AAG-1-1// . . . *11 | Exact | 16 | 134 | 42 | 1.68 | 0.546 | 7.34 | 0.041 |
| Example 36 | 36 | isomiR | mir-150 | Mature 5′ sub | 21 | 7252 | 2372 | 1.61 | 0.738 | 11.13 | 0.023 |
| Example 37 | 37 | isomiR | mir-146b | Mature 5′ super | 24 | 255 | 85 | 1.53 | 0.850 | 6.54 | 0.001 |
| Example 38 | 38 | tRF | tRNA-Glu-CTC-1-l// . . . *12 | Exact | 30 | 86 | 28 | 1.63 | 0.771 | 5.99 | 0.001 |
| Example 39 | 39 | isomiR | mir-223 | Mature 3′ sub | 20 | 2960 | 1043 | 1.86 | 0.792 | 8.85 | 0.002 |
| Example 40 | 40 | isomiR | mir-145 | Mature 5′ super | 24 | 116 | 41 | 1.50 | 0.790 | 5.48 | 0.005 |
| Example 41 | 41 | isomiR | mir-186 | Mature 5′ sub | 21 | 322 | 112 | 1.53 | 0.921 | 7.74 | 0.000 |

TABLE 2-3

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 42 | 42 | miRNA | mir-365a//mir-365b | Mature 3′ | 22 | 169 | 61 | 1.29 | 0.808 | 6.55 | 0.005 |
| Example 43 | 43 | isomiR | mir-223 | Mature 3′ super | 23 | 167 | 62 | 1.43 | 0.700 | 6.90 | 0.012 |
| Example 44 | 44 | isomiR | mir-192 | Mature 5′ sub | 20 | 344 | 130 | 1.40 | 0.608 | 7.93 | 0.033 |
| Example 45 | 45 | tRF | tRNA-Gly-GCC- | Exact | 33 | 131 | 50 | 1.38 | 0.733 | 4.10 | 0.047 |
| | | | 2-1// . . . *13 | | | | | | | | |
| Example 46 | 46 | miRNA | mir-17 | Mature 5′ | 23 | 1458 | 590 | 1.39 | 0.888 | 9.88 | 0.000 |
| Example 47 | 47 | isomiR | mir-339 | Mature 5′ sub | 19 | 156 | 64 | 1.29 | 0.748 | 5.61 | 0.011 |
| Example 48 | 48 | isomiR | mir-223 | Mature 3′ sub | 21 | 6065 | 2585 | 1.23 | 0.763 | 11.58 | 0.007 |
| Example 49 | 49 | isomiR | mir-223 | Mature 3′ sub | 21 | 10177 | 4407 | 1.21 | 0.754 | 11.30 | 0.010 |
| Example 50 | 50 | isomiR | mir-30c-2//mir-30c-1 | Mature 5′ sub | 22 | 86 | 36 | 1.26 | 0.754 | 5.77 | 0.007 |
| Example 51 | 51 | isomiR | mir-1307 | Mature 3′ super | 23 | 46 | 20 | 1.18 | 0.767 | 5.33 | 0.003 |
| Example 52 | 52 | miRNA | mir-29c | Mature 3′ | 22 | 704 | 310 | 1.50 | 0.796 | 8.76 | 0.002 |
| Example 53 | 53 | isomiR | mir-223 | Mature 3′ sub | 20 | 517 | 232 | 1.16 | 0.738 | 6.16 | 0.016 |
| Example 54 | 54 | isomiR | mir-223 | Mature 3′ super | 24 | 94 | 42 | 1.17 | 0.617 | 6.32 | 0.047 |
| Example 55 | 55 | isomiR | mir-30b | Mature 5′ sub | 21 | 93 | 41 | 1.19 | 0.742 | 6.27 | 0.008 |
| Example 56 | 56 | isomiR | mir-766 | Mature 3 sub | 21 | 78 | 36 | 1.11 | 0.733 | 5.34 | 0.012 |
| Example 57 | 57 | isomiR | mir-26b | Mature 3′ sub | 21 | 37 | 17 | 1.11 | 0.744 | 4.02 | 0.017 |
| Example 58 | 58 | tRF | tRNA-Gly-CCC- | Exact | 22 | 310 | 140 | 1.14 | 0.631 | 9.06 | 0.037 |
| | | | 1-1// . . . *14 | | | | | | | | |
| Example 59 | 59 | miRNA | let-7d | Mature 3′ | 22 | 103 | 48 | 1.12 | 0.802 | 6.86 | 0.003 |
| Example 60 | 60 | tRF | tRNA-Gly-CCC- | Exact | 25 | 415 | 191 | 1.12 | 0.617 | 9.15 | 0.053 |
| | | | 1-1// . . . *15 | | | | | | | | |
| Example 61 | 61 | isomiR | mir-30d | Mature 5′ sub | 19 | 144 | 69 | 1.07 | 0.721 | 6.82 | 0.016 |
| Example 62 | 62 | miRNA | mir-505 | Mature 3′ | 22 | 55 | 26 | 1.08 | 0.767 | 5.34 | 0.007 |
| Example 63 | 63 | isomiR | mir-93 | Mature 5′ sub | 22 | 61 | 28 | 1.13 | 0.767 | 4.66 | 0.032 |
| Example 64 | 64 | isomiR | mir-30e | Mature 5′ super | 23 | 817 | 384 | 1.09 | 0.867 | 9.44 | 0.000 |

TABLE 2-4

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 65 | 65 | precursor | mir-16-1// | precursor miRNA | 16 | 114 | 54 | 1.09 | 0.740 | 6.33 | 0.012 |
| | | | mir-16-2 | | | | | | | | |
| Example 66 | 66 | miRNA | mir-193a | Mature 5 | 22 | 245 | 121 | 1.19 | 0.771 | 7.30 | 0.006 |
| Example 67 | 67 | isomiR | mir-320a | Mature 3′ super | 25 | 46 | 22 | 1.07 | 0.717 | 4.37 | 0.019 |
| Example 68 | 68 | isomiR | mir-29b-1// | Mature 3′ sub | 21 | 187 | 93 | 1.01 | 0.650 | 7.06 | 0.023 |
| | | | mir-29b-2 | | | | | | | | |
| Example 69 | 69 | isomiR | mir-142 | Mature 5′ sub/super | 22 | 458 | 242 | 0.92 | 0.717 | 8.13 | 0.043 |
| Example 70 | 70 | isomiR | mir-142 | Mature 5′ sub/super | 21 | 117 | 60 | 0.97 | 0.731 | 5.33 | 0.045 |
| Example 71 | 71 | miRNA | mir-744 | Mature 5′ | 22 | 131 | 69 | 0.92 | 0.758 | 6.31 | 0.012 |
| Example 72 | 72 | isomiR | mir-200b | Mature 3′ sub | 21 | 2 | 27 | −3.48 | 0.900 | 2.69 | 0.000 |
| Example 73 | 73 | isomiR | mir-181b-1// | Mature 5′ sub | 19 | 20 | 203 | −5.29 | 0.946 | 5.09 | 0.000 |
| | | | mir-181b-2 | | | | | | | | |
| Example 74 | 74 | isomiR | mir-200a | Mature 3′ sub | 18 | 5 | 47 | −4.05 | 0.950 | 4.13 | 0.000 |
| Example 75 | 75 | isomiR | mir-181b-1// | Mature 5′ sub | 18 | 37 | 296 | −5.43 | 0.942 | 5.40 | 0.000 |
| | | | mir-181b-2 | | | | | | | | |
| Example 76 | 76 | isomiR | mir-181b-1// | Mature 5′ sub | 20 | 79 | 583 | −5.95 | 0.917 | 5.40 | 0.000 |
| | | | mir-181b-2 | | | | | | | | |
| Example 77 | 77 | miRNA | mir-340 | Mature 3′ | 22 | 312 | 2209 | −7.02 | 0.938 | 8.82 | 0.000 |
| Example 78 | 78 | isomiR | mir-181b-1// | Mature 5′ sub | 21 | 33 | 223 | −4.97 | 0.921 | 5.40 | 0.000 |
| | | | mir-181b-2 | | | | | | | | |
| Example 79 | 79 | miRNA | mir-378e | Mature 3′ | 19 | 5 | 33 | −3.37 | 0.865 | 2.69 | 0.000 |
| Example 80 | 80 | precursor | mir-181b-1// | precursor miRNA | 17 | 17 | 100 | −4.43 | 0.925 | 5.80 | 0.000 |
| | | | mir-181b-2 | | | | | | | | |
| Example 81 | 81 | isomiR | mir-145 | Mature 5′ sub | 19 | 6 | 32 | −3.42 | 0.867 | 3.21 | 0.000 |
| Example 82 | 82 | precursor | mir-181b-1// | precursor miRNA | 16 | 12 | 71 | −3.96 | 0.873 | 4.61 | 0.000 |
| | | | mir-181b-2 | | | | | | | | |

TABLE 2-5

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 83 | 83 | isomiR | mir-181b-1//mir-181 | Mature 5′ sub | 22 | 64 | 343 | −4.91 | 0.925 | 6.37 | 0.000 |
| | | | b-2 | | | | | | | | |
| Example 84 | 84 | isomiR | mir-451a | Mature 5′ sub | 18 | 7 | 33 | −3.31 | 0.942 | 3.81 | 0.000 |
| Example 85 | 85 | isomiR | mir-29b-1//mir-29b-2 | Mature 3′ sub | 22 | 15 | 69 | −3.75 | 0.863 | 2.69 | 0.000 |
| Example 86 | 86 | isomiR | mir-451a | Mature 5′ sub | 17 | 13 | 55 | −2.90 | 0.913 | 4.67 | 0.000 |
| Example 87 | 87 | precursor | mir-181b-1//mir-181 | precursor | 15 | 9 | 38 | −3.16 | 0.844 | 4.63 | 0.000 |
| | | | b-2 | miRNA | | | | | | | |
| Example 88 | 88 | isomiR | mir-144 | Mature 3′ sub | 17 | 20 | 75 | −2.55 | 0.854 | 5.64 | 0.002 |
| Example 89 | 89 | isomiR | mir-451a | Mature 5′ | 18 | 16 | 55 | −2.15 | 0.850 | 5.48 | 0.009 |
| | | | sub/super | | | | | | | | |
| Example 90 | 90 | isomiR | mir-451a | Mature 5′ sub | 19 | 14 | 46 | −2.46 | 0.850 | 4.58 | 0.000 |
| Example 91 | 91 | miRNA | let-7c | Mature 5′ | 22 | 11 | 35 | −2.24 | 0.821 | 3.18 | 0.002 |
| Example 92 | 92 | isomiR | mir-16-2 | Mature 3′ | 20 | 119 | 362 | −1.87 | 0.967 | 7.97 | 0.000 |
| | | | | sub/super | | | | | | | |
| Example 93 | 93 | isomiR | mir-451a | Mature 5′ super | 25 | 11282 | 31795 | −1.49 | 0.671 | 14.65 | 0.043 |
| Example 94 | 94 | isomiR | mir-486-1 | Mature 5′ super | 23 | 15 | 42 | −1.48 | 0.796 | 4.18 | 0.020 |
| Example 95 | 95 | isomiR | mir-126 | Mature 3′ sub | 20 | 29 | 80 | −1.87 | 0.842 | 5.55 | 0.006 |
| Example 96 | 96 | isomiR | mir-363 | Mature 3′ sub | 19 | 15 | 38 | −1.39 | 0.802 | 3.98 | 0.022 |
| Example 97 | 97 | isomiR | mir-574 | Mature 5′ sub | 21 | 22 | 56 | −2.16 | 0.829 | 5.18 | 0.001 |
| Example 98 | 98 | miRNA | let-7b | Mature 5′ | 22 | 1771 | 4518 | −1.28 | 0.817 | 10.67 | 0.001 |
| Example 99 | 99 | miRNA | mir-144 | Mature 3′ | 20 | 660 | 1687 | −1.35 | 0.771 | 9.97 | 0.028 |
| Example 100 | 100 | isomiR | mir-574 | Mature 3′ sub | 21 | 17 | 43 | −2.04 | 0.846 | 4.22 | 0.000 |
| Example 101 | 101 | isomiR | let-7b | Mature 5′ sub | 21 | 1614 | 3915 | −1.50 | 0.900 | 10.98 | 0.000 |
| Example 102 | 102 | isomiR | mir-103a-2//mir- | Mature 3′ sub | 19 | 648 | 1544 | −1.06 | 0.717 | 10.94 | 0.008 |
| | | | 103a-1//mir-107 | | | | | | | | |
| Example 103 | 103 | isomiR | mir-126 | Mature 3′ sub | 21 | 301 | 713 | −1.56 | 0.854 | 8.66 | 0.002 |
| Example 104 | 104 | isomiR | mir-451a | Mature 5′ super | 24 | 19 | 43 | −1.18 | 0.738 | 4.01 | 0.072 |
| Example 105 | 105 | miRNA | mir-106b | Mature 5′ | 21 | 670 | 1524 | −1.13 | 0.888 | 10.36 | 0.001 |

TABLE 2-6

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 106 | 106 | miRNA | let-7i | Mature 5′ | 22 | 107 | 247 | −1.20 | 0.804 | 7.46 | 0.014 |
| Example 107 | 107 | precursor | mir-451a | precursor | 15 | 49 | 106 | −1.11 | 0.783 | 6.13 | 0.036 |
| | | | | miRNA | | | | | | | |
| Example 108 | 108 | isomiR | mir-425 | Mature 5′ sub | 19 | 14 | 31 | −1.13 | 0.819 | 4.10 | 0.031 |
| Example 109 | 109 | isomiR | mir-16-2 | Mature 3′ sub | 20 | 15 | 33 | −1.82 | 0.754 | 4.51 | 0.003 |
| Example 110 | 110 | miRNA | mir-139 | Mature 5′ | 23 | 69 | 155 | −1.18 | 0.771 | 7.08 | 0.024 |
| Example 111 | 111 | isomiR | mir-451a | Mature 5′ super | 23 | 38 | 80 | −1.10 | 0.715 | 6.35 | 0.047 |
| Example 112 | 112 | isomiR | mir-18a | Mature 5′ sub | 21 | 138 | 296 | −1.10 | 0.767 | 7.79 | 0.030 |
| Example 113 | 113 | miRNA | mir-126 | Mature 3′ | 22 | 335 | 706 | −1.23 | 0.833 | 8.69 | 0.004 |
| Example 114 | 114 | isomiR | mir-550a-1//mir-550a- | Mature 3′ sub | 21 | 63 | 133 | −1.50 | 0.775 | 6.23 | 0.005 |
| | | | 2//mir-550a-3 | | | | | | | | |
| Example 115 | 115 | isomiR | mir-142 | Mature 3′ sub | 22 | 181 | 222 | −0.30 | 0.504 | 8.05 | 0.548 |
| Example 116 | 116 | isomiR | mir-142 | Mature 3′ sub | 21 | 156 | 135 | 0.21 | 0.517 | 5.74 | 0.577 |
| Example 122 | 119 | MiscRNA | ENST00000363745. | Exact | 28 | 484 | 40 | 6.44 | 0.936 | 5.79 | 0.000 |
| | | | 1// . . . *16 | | | | | | | | |
| Example 123 | 120 | MiscRNA | ENST00000364600. | Exact | 31 | 1504 | 95 | 6.35 | 0.951 | 8.41 | 0.000 |
| | | | 1// . . . *17 | | | | | | | | |
| Example 124 | 121 | miRNA | mir-221 | Mature 3′ | 23 | 457 | 32 | 5.92 | 0.923 | 7.09 | 0.000 |
| Example 125 | 122 | miRNA | mir-374b | Mature 5′ | 22 | 465 | 44 | 5.44 | 0.931 | 7.50 | 0.000 |
| Example 126 | 123 | isomiR | mir-130a | Mature 3′ super | 23 | 293 | 32 | 5.43 | 0.904 | 6.27 | 0.000 |
| Example 127 | 124 | miRNA | mir-340 | Mature 5′ | 22 | 495 | 47 | 5.40 | 0.932 | 7.23 | 0.000 |
| Example 128 | 125 | miRNA | mir-199a-1//mir-199a- | Mature 3′ | 22 | 2387 | 161 | 5.21 | 0.958 | 9.23 | 0.000 |
| | | | 2//mir-199b | | | | | | | | |
| Example 129 | 126 | isomiR | mir-23a | Mature 3′ super | 23 | 927 | 92 | 4.98 | 0.914 | 8.22 | 0.000 |
| Example 130 | 127 | miRNA | mir-335 | Mature 5′ | 23 | 632 | 89 | 4.84 | 0.949 | 7.50 | 0.000 |
| Example 131 | 128 | miRNA | mir-130a | Mature 3′ | 22 | 3873 | 417 | 3.70 | 0.962 | 10.40 | 0.000 |
| Example 132 | 129 | isomiR | mir-584 | Mature 5′ sub | 21 | 619 | 121 | 3.38 | 0.897 | 8.04 | 0.000 |
| Example 133 | 130 | MiscRNA | ENST00000363745. | Exact | 26 | 13226 | 2207 | 2.72 | 0.908 | 12.82 | 0.000 |
| | | | 1// . . . *18 | | | | | | | | |

TABLE 2-7

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 134 | 131 | miRNA | mir−26a-1// | Mature 5′ | 22 | 5509 | 853 | 2.66 | 0.931 | 11.03 | 0.000 |
| | | | mir−26a-2 | | | | | | | | |
| Example 135 | 132 | MiscRNA | ENST00000364600. | Exact | 32 | 151813 | 17667 | 2.56 | 0.932 | 15.67 | 0.000 |
| | | | 1// . . . *17 | | | | | | | | |
| Example 136 | 133 | isomiR | mir-23a | Mature 3′ super | 22 | 12447 | 2197 | 2.19 | 0.947 | 12.60 | 0.000 |
| Example 137 | 134 | miRNA | mir-146a | Mature 5′ | 22 | 2236 | 549 | 2.05 | 0.915 | 10.03 | 0.000 |
| Example 138 | 135 | miRNA | mir-191 | Mature 5′ | 23 | 3434 | 726 | 2.04 | 0.926 | 10.19 | 0.000 |
| Example 139 | 136 | MiscRNA | ENST00000364600. | Exact | 31 | 106642 | 25718 | 2.02 | 0.939 | 15.70 | 0.000 |
| | | | 1// . . . *17 | | | | | | | | |
| Example 140 | 137 | miRNA | mir-92a-1// | Mature 3 | 22 | 2418 | 8103 | −2.07 | 0.941 | 11.90 | 0.000 |
| | | | mir-92a-2 | | | | | | | | |
| Example 141 | 138 | isomiR | let-7b | Mature 5′ sub | 20 | 416 | 1273 | −2.15 | 0.901 | 9.56 | 0.000 |
| Example 142 | 139 | isomiR | mir-451a | Mature 5′ sub | 21 | 13722 | 36210 | −2.15 | 0.905 | 14.34 | 0.000 |
| Example 143 | 140 | isomiR | mir-30e | Mature 5′ | 23 | 414 | 1361 | −2.21 | 0.972 | 9.67 | 0.000 |
| | | | | sub/super | | | | | | | |
| Example 144 | 141 | isomiR | let-7g | Mature 5′ sub | 21 | 875 | 3513 | −2.28 | 0.972 | 10.48 | 0.000 |
| Example 145 | 142 | miRNA | mir-486-1// | Mature 5′ | 22 | 2037 | 7408 | −2.44 | 0.935 | 11.36 | 0.000 |
| | | | mir-486-2 | | | | | | | | |
| Example 146 | 143 | isomiR | mir-16-1//mir-16-2 | Mature 5′ sub | 20 | 2087 | 8031 | −2.47 | 0.977 | 12.12 | 0.000 |
| Example 147 | 144 | isomiR | mir-451a | Mature 5′ sub | 20 | 7902 | 30578 | −2.61 | 0.957 | 14.22 | 0.000 |
| Example 148 | 145 | isomiR | mir-185 | Mature 5′ sub | 21 | 595 | 2886 | −2.67 | 0.978 | 10.52 | 0.000 |
| Example 149 | 146 | isomiR | let-7a-1//let-7a-2// | Mature 5′ sub | 20 | 633 | 3159 | −2.67 | 0.975 | 10.97 | 0.000 |
| | | | let-7a-3 | | | | | | | | |
| Example 150 | 147 | isomiR | mir-92a-1// | Mature 3′ sub | 21 | 247 | 882 | −2.73 | 0.904 | 8.30 | 0.000 |
| | | | mir-92a-2 | | | | | | | | |
| Example 151 | 148 | isomiR | mir−25 | Mature 3′ sub | 21 | 214 | 916 | −2.86 | 0.961 | 8.79 | 0.000 |
| Example 152 | 149 | isomiR | mir-16-2 | Mature 3′ | 21 | 159 | 708 | −2.87 | 0.921 | 8.60 | 0.000 |
| | | | | sub/super | | | | | | | |

TABLE 2-8

| | | | | | | Average in | Average | | | | |
|---|---|---|---|---|---|---|---|---|---|---|---|
| | SEQ | | | | Length | head and | in | | | Cut-off | |
| | ID | | | | (nucleo- | neck cancer | healthy | Log2 | | value | |
| Example | NO: | Class | Archetype | Type | tides) | patients | subjects | FC | AUC | (Log2) | p-value |
| Example 153 | 150 | isomiR | let-7f-1//let-7f-2 | Mature 5′ sub | 20 | 253 | 1372 | −2.98 | 0.956 | 9.04 | 0.000 |
| Example 154 | 151 | isomiR | mir-25 | Mature 3′ sub | 20 | 117 | 538 | −3.01 | 0.931 | 7.93 | 0.000 |
| Example 155 | 152 | isomiR | mir-425 | Mature 5′ sub | 21 | 147 | 634 | −3.15 | 0.945 | 8.53 | 0.000 |
| Example 156 | 153 | isomiR | mir-423 | Mature 5′ sub | 21 | 588 | 2940 | −3.15 | 0.962 | 10.52 | 0.000 |
| Example 157 | 154 | isomiR | mir-484 | Mature 5′ sub | 21 | 635 | 3996 | −3.27 | 0.966 | 10.23 | 0.000 |
| Example 158 | 155 | isomiR | mir-486-1//mir-486-2 | Mature 5 sub | 21 | 2876 | 17383 | −3.32 | 0.956 | 12.95 | 0.000 |
| Example 159 | 156 | isomiR | mir-486-1//mir-486-2 | Mature 5′ sub | 20 | 280 | 1771 | −3.48 | 0.952 | 9.47 | 0.000 |
| Example 160 | 157 | isomiR | let-7i | Mature 5′ sub | 21 | 460 | 3333 | −3.61 | 0.969 | 10.35 | 0.000 |
| Example 161 | 158 | isomiR | let-7d | Mature 5′ sub | 20 | 116 | 685 | −3.75 | 0.943 | 8.46 | 0.000 |
| Example 162 | 159 | isomiR | mir-486-1//mir-486-2 | Mature 5′ sub | 17 | 20 | 207 | −4.08 | 0.917 | 6.00 | 0.000 |
| Example 163 | 160 | isomiR | let-7i | Mature 5′ sub | 20 | 89 | 857 | −4.36 | 0.981 | 8.54 | 0.000 |
| Example 164 | 161 | isomiR | mir-484 | Mature 5′ sub | 20 | 43 | 497 | −4.85 | 0.964 | 7.76 | 0.000 |
| Example 165 | 162 | LincRNA | ENST00000627566.1 | Exact | 15 | 8 | 349 | −7.39 | 0.986 | 3.97 | 0.000 |
| Example 167 | 117 | miRNA | mir-339 | Mature 3′ | 23 | 4 | 8 | 0.55 | 0.625 | 11.4 | 0.413 |
| Example 168 | 118 | miRNA | mir-17 | Mature 3′ | 22 | 17 | 8 | −0.96 | 0.621 | 17.17 | 0.250 |

TABLE 2-9

| | SEQ ID | | Archetype | |
|---|---|---|---|---|
| Example | NOs: | Class | and Type | Fold Change |
| Example | 115, 116 | isomiRNA | mir-142 Mature | Before surgery: −2.1 |
| 117 | | | 3’ sub | After surgery: −2.4 |

TABLE 2-10

| | SEQ ID | | Archetype | Cut-off | AUC |
|---|---|---|---|---|---|
| Examples | NOs: | Class | and Type | value | value |
| Example | 11 and | miRNA | mir-150-5p and | 4.83 | 0.97628 |
| 118 | 30 | | mir-146b-5p | | |
| Example | 11 and | miRNA | mir-150-5p and | 5.05 | 0.96443 |
| 119 | 26 | | mir-29a-3p | | |
| Example | 11 and | miRNA | mir-150-5p and | 4.82 | 0.94071 |
| 120 | 117 | | mir-339-3p | | |
| Example | 30 and | miRNA | mir-146b-5p and | 5.05 | 0.91406 |
| 121 | 118 | | mir-17-3p | | |
| Example | 157 and | isomiR, | let-7i Mature 5’ sub and | 3.03 | 0.967 |
| 166 | 162 | LincRNA | ENST00000627566.1 | | |

As seen in these results, the abundance of the miRNAs or the like represented by SEQ ID NOs: 1 to 71 and 118 to 136 was significantly higher in the patients with head and neck cancer than that in the healthy subjects, and the miRNAs or the like represented by SEQ ID NOs: 72 to 117 and 137 to 162 was significantly lower in the patients with head and neck cancer than in the healthy subjects. It was indicated that head and neck cancer was able to be detected with high accuracy by the method of the present invention (Examples Ito 116, 122 to 165, and 167 to 168).

Moreover, the result presented in Table 2-9 showed that the FC (fold change) in the abundance of the isomiR represented by either SEQ ID NO: 115 or SEQ ID NO: 116 was changed before and after surgery for tongue cancer, indicating that the isomiRs can be used to assess the success or failure of the surgery. Furthermore, the result presented in Table 2-10 showed that the combinations of the miRNAs represented by SEQ ID NOs: 11 and 30, SEQ ID NOs: 11 and 26, SEQ ID NOs: 11 and 117, SEQ ID NOs: 30 and 118, and SEQ ID NOs: 157 and 162 had an AUC value ranging from 0.91406 to 0.97628, indicating that even early tongue cancer can be detected by using any of the combinations.

1. A method of assisting the detection of head and neck cancer, using as an index the abundance of at least one of miRNAs, isoform miRNAs (isomiRs), precursor miRNAs, transfer RNA fragments (tRFs), or non-coding RNA fragments (LincRNAs or MiscRNAs) contained in a test sample isolated from a living body, whose nucleotide sequence is represented by any one of SEQ ID NOs: 162, 160, 145, 143, 146, 140, 141, 1 to 139, 142, 144, 147 to 159, and 161, wherein a higher abundance of at least one of the miRNAs, isomiRs, precursor miRNAs, transfer RNA fragments, or non-coding RNA fragments whose nucleotide sequence is represented by any one of SEQ ID NOs: 1 to 71 and 118 to 136 than that of healthy subjects or a lower abundance of at least one of the miRNAs, isomiRs, precursor miRNAs, transfer RNA fragments, or non-coding RNA fragments whose nucleotide sequence is represented by any one of SEQ ID NOs: 72 to 117 and 137 to 162 than that of healthy subjects indicates a higher likelihood of having head and neck cancer.

2. The method according to claim 1, wherein the abundance of at least one of miRNAs, isoform miRNAs (isomiRs), precursor miRNAs, transfer RNA fragments (tRFs), or non-coding RNA fragments (LincRNAs or MiscRNAs) whose nucleotide sequence is represented by any one of SEQ ID NOs: 162, 160, 145, 143, 146, 140, and 141 is used as an index.

3. The method according to claim 1, wherein the abundance of at least one of miRNAs, isoform miRNAs (isomiRs), precursor miRNAs, or transfer RNA fragments whose nucleotide sequence is represented by any one of SEQ ID NOs: 92, 2, 74, 73, 75, 84, 32, 77, 18, 1, 3 to 31, 33 to 72, 76, 78 to 83, 85 to 91, and 93 to 116 is used as an index.

4. The method according to claim 3, wherein the abundance of an isomiR whose nucleotide sequence is represented by SEQ ID NO: 115 or 116 is used as an index.

5. The method according to claim 3, wherein the abundance of a miRNA whose nucleotide sequence is represented by SEQ ID NO: 11 and a miRNA whose nucleotide sequence is represented by SEQ ID NO: 30 is used as an index.

6. The method according to claim 3, wherein the abundance of a miRNA whose nucleotide sequence is represented by SEQ ID NO: 11 and a miRNA whose nucleotide sequence is represented by SEQ ID NO: 26 is used as an index.

7. The method according to claim 3, wherein the abundance of a miRNA whose nucleotide sequence is represented by SEQ ID NO: 11 and a miRNA whose nucleotide sequence is represented by SEQ ID NO: 117 is used as an index.

8. The method according to claim 3, wherein the abundance of a miRNA whose nucleotide sequence is represented by SEQ ID NO: 30 and a miRNA whose nucleotide sequence is represented by SEQ ID NO: 118 is used as an index.

9. The method according to claim 1, wherein the abundance of a miRNA whose nucleotide sequence is represented by SEQ ID NO: 157 and a miRNA whose nucleotide sequence is represented by SEQ ID NO: 162 is used as an index.

10. The method according to claim 1, wherein the head and neck cancer is tongue cancer.


SEQUENCE LISTING (162 sequences)

SEQ ID NO: 1 - RNA, 30 nt, Homo sapiens
gcauuggugguucagugguagaauucucgc

SEQ ID NO: 2 - RNA, 28 nt, Homo sapiens
cggauagcucagucgguagagcaucaga

SEQ ID NO: 3 - RNA, 32 nt, Homo sapiens
ucccugguggucuagugguuaggauucggcgc

SEQ ID NO: 4 - RNA, 31 nt, Homo sapiens
ggcucguuggucuagggguaugauucucggu

SEQ ID NO: 5 - RNA, 31 nt, Homo sapiens
gcccggauagcucagucgguagagcaucaga

SEQ ID NO: 6 - RNA, 33 nt, Homo sapiens
agcagaguggcgcagcggaagcgugcugggccc

SEQ ID NO: 7 - RNA, 31 nt, Homo sapiens
gcccggcuagcucagucgguagagcauggga

SEQ ID NO: 8 - RNA, 31 nt, Homo sapiens
agcagaguggcgcagcggaagcgugcugggc

SEQ ID NO: 9 - RNA, 21 nt, Homo sapiens
auggcacugguagaauucacu

SEQ ID NO: 10 - RNA, 17 nt, Homo sapiens
ugucaguuugucaaaua

SEQ ID NO: 11 - RNA, 22 nt, Homo sapiens
ucucccaacccuuguaccagug

SEQ ID NO: 12 - RNA, 24 nt, Homo sapiens
ugucaguuugucaaauaccccaag

SEQ ID NO: 13 - RNA, 28 nt, Homo sapiens
cggcuagcucagucgguagagcauggga

SEQ ID NO: 14 - RNA, 23 nt, Homo sapiens
ucucccaacccuuguaccagugc

SEQ ID NO: 15 - RNA, 19 nt, Homo sapiens
ucucccaacccuuguacca

SEQ ID NO: 16 - RNA, 30 nt, Homo sapiens
ggcucguuggucuagggguaugauucucgc

SEQ ID NO: 17 - RNA, 23 nt, Homo sapiens
ugagaacugaauuccauaggcug

SEQ ID NO: 18 - RNA, 30 nt, Homo sapiens
agcagaguggcgcagcggaagcgugcuggg

SEQ ID NO: 19 - RNA, 24 nt, Homo sapiens
ucccccaggugugauucugauuug

SEQ ID NO: 20 - RNA, 21 nt, Homo sapiens
ucaguuugucaaauaccccaa

SEQ ID NO: 21 - RNA, 15 nt, Homo sapiens
ugucaguuugucaaa

SEQ ID NO: 22 - RNA, 16 nt, Homo sapiens
ugucaguuugucaaau

SEQ ID NO: 23 - RNA, 20 nt, Homo sapiens
ugagaacugaauuccauggg

SEQ ID NO: 24 - RNA, 20 nt, Homo sapiens
ucucccaacccuuguaccag

SEQ ID NO: 25 - RNA, 18 nt, Homo sapiens
ugucaguuugucaaauac

SEQ ID NO: 26 - RNA, 22 nt, Homo sapiens
uagcaccaucugaaaucgguua

SEQ ID NO: 27 - RNA, 20 nt, Homo sapiens
ucaguuugucaaauacccca

SEQ ID NO: 28 - RNA, 23 nt, Homo sapiens
ucccuguccuccaggagcucacg

SEQ ID NO: 29 - RNA, 23 nt, Homo sapiens
ugucaguuugucaaauaccccaa

SEQ ID NO: 30 - RNA, 22 nt, Homo sapiens
ugagaacugaauuccauaggcu

SEQ ID NO: 31 - RNA, 21 nt, Homo sapiens
uaaugccccuaaaaauccuua

SEQ ID NO: 32 - RNA, 22 nt, Homo sapiens
cagugguuuuacccuaugguag

SEQ ID NO: 33 - RNA, 22 nt, Homo sapiens
ugucaguuugucaaauacccca

SEQ ID NO: 34 - RNA, 22 nt, Homo sapiens
gucaguuugucaaauaccccaa

SEQ ID NO: 35 - RNA, 16 nt, Homo sapiens
gguagcguggccgagc

SEQ ID NO: 36 - RNA, 21 nt, Homo sapiens
ucucccaacccuuguaccagu

SEQ ID NO: 37 - RNA, 24 nt, Homo sapiens
ugagaacugaauuccauaggcugu

SEQ ID NO: 38 - RNA, 30 nt, Homo sapiens
ucccugguggucuagugguuaggauucggc

SEQ ID NO: 39 - RNA, 20 nt, Homo sapiens
ugucaguuugucaaauaccc

SEQ ID NO: 40 - RNA, 24 nt, Homo sapiens
guccaguuuucccaggaaucccuu

SEQ ID NO: 41 - RNA, 21 nt, Homo sapiens
caaagaauucuccuuuugggc

SEQ ID NO: 42 - RNA, 22 nt, Homo sapiens
uaaugccccuaaaaauccuuau

SEQ ID NO: 43 - RNA, 23 nt, Homo sapiens
gugucaguuugucaaauacccca

SEQ ID NO: 44 - RNA, 20 nt, Homo sapiens
ugaccuaugaauugacagcc

SEQ ID NO: 45 - RNA, 33 nt, Homo sapiens
gcauuggugguucagugguagaauucucgccug

SEQ ID NO: 46 - RNA, 23 nt, Homo sapiens
caaagugcuuacagugcagguag

SEQ ID NO: 47 - RNA, 19 nt, Homo sapiens
ucccuguccuccaggagcu

SEQ ID NO: 48 - RNA, 21 nt, Homo sapiens
ugucaguuugucaaauacccc

SEQ ID NO: 49 - RNA, 21 nt, Homo sapiens
gucaguuugucaaauacccca

SEQ ID NO: 50 - RNA, 22 nt, Homo sapiens
uguaaacauccuacacucucag

SEQ ID NO: 51 - RNA, 23 nt, Homo sapiens
acucggcguggcgucggucgugg

SEQ ID NO: 52 - RNA, 22 nt, Homo sapiens
uagcaccauuugaaaucgguua

SEQ ID NO: 53 - RNA, 20 nt, Homo sapiens
gucaguuugucaaauacccc

SEQ ID NO: 54 - RNA, 24 nt, Homo sapiens
gugucaguuugucaaauaccccaa

SEQ ID NO: 55 - RNA, 21 nt, Homo sapiens
uguaaacauccuacacucagc

SEQ ID NO: 56 - RNA, 21 nt, Homo sapiens
acuccagccccacagccucag

SEQ ID NO: 57 - RNA, 21 nt, Homo sapiens
ccuguucuccauuacuuggcu

SEQ ID NO: 58 - RNA, 22 nt, Homo sapiens
gcauuggugguucagugguaga

SEQ ID NO: 59 - RNA, 22 nt, Homo sapiens
cuauacgaccugcugccuuucu

SEQ ID NO: 60 - RNA, 25 nt, Homo sapiens
gcauuggugguucagugguagaauu

SEQ ID NO: 61 - RNA, 19 nt, Homo sapiens
uguaaacauccccgacugg

SEQ ID NO: 62 - RNA, 22 nt, Homo sapiens
cgucaacacuugcugguuuccu

SEQ ID NO: 63 - RNA, 22 nt, Homo sapiens
aaagugcuguucgugcagguag

SEQ ID NO: 64 - RNA, 23 nt, Homo sapiens
uguaaacauccuugacuggaagc

SEQ ID NO: 65 - RNA, 16 nt, Homo sapiens
uagcagcacguaaaua

SEQ ID NO: 66 - RNA, 22 nt, Homo sapiens
ugggucuuugcgggcgagauga

SEQ ID NO: 67 - RNA, 25 nt, Homo sapiens
aaaagcuggguugagagggcgaaaa

SEQ ID NO: 68 - RNA, 21 nt, Homo sapiens
uagcaccauuugaaaucagug

SEQ ID NO: 69 - RNA, 22 nt, Homo sapiens
cccauaaaguagaaagcacuac

SEQ ID NO: 70 - RNA, 21 nt, Homo sapiens
cccauaaaguagaaagcacua

SEQ ID NO: 71 - RNA, 22 nt, Homo sapiens
ugcggggcuagggcuaacagca

SEQ ID NO: 72 - RNA, 21 nt, Homo sapiens
aauacugccugguaaugauga

SEQ ID NO: 73 - RNA, 19 nt, Homo sapiens
uucauugcugucggugggu

SEQ ID NO: 74 - RNA, 18 nt, Homo sapiens
acugucugguaacgaugu

SEQ ID NO: 75 - RNA, 18 nt, Homo sapiens
ucauugcugucggugggu

SEQ ID NO: 76 - RNA, 20 nt, Homo sapiens
auucauugcugucggugggu

SEQ ID NO: 77 - RNA, 22 nt, Homo sapiens
uccgucucaguuacuuuauagc

SEQ ID NO: 78 - RNA, 21 nt, Homo sapiens
cauucauugcugucggugggu

SEQ ID NO: 79 - RNA, 19 nt, Homo sapiens
acuggacuuggagucagga

SEQ ID NO: 80 - RNA, 17 nt, Homo sapiens
cauugcugucggugggu

SEQ ID NO: 81 - RNA, 19 nt, Homo sapiens
aguuuucccaggaaucccu

SEQ ID NO: 82 - RNA, 16 nt, Homo sapiens
auugcugucggugggu

SEQ ID NO: 83 - RNA, 22 nt, Homo sapiens
acauucauugcugucggugggu

SEQ ID NO: 84 - RNA, 18 nt, Homo sapiens
cguuaccauuacugaguu

SEQ ID NO: 85 - RNA, 22 nt, Homo sapiens
agcaccauuugaaaucaguguu

SEQ ID NO: 86 - RNA, 17 nt, Homo sapiens
guuaccauuacugaguu

SEQ ID NO: 87 - RNA, 15 nt, Homo sapiens
uugcugucggugggu

SEQ ID NO: 88 - RNA, 17 nt, Homo sapiens
uacaguauagaugaugu

SEQ ID NO: 89 - RNA, 18 nt, Homo sapiens
guuaccauuacugaguuu

SEQ ID NO: 90 - RNA, 19 nt, Homo sapiens
accguuaccauuacugagu

SEQ ID NO: 91 - RNA, 22 nt, Homo sapiens
ugagguaggagguuguauaguu

SEQ ID NO: 92 - RNA, 20 nt, Homo sapiens
accaauauuacugugcugcu

SEQ ID NO: 93 - RNA, 25 nt, Homo sapiens
aaaccguuaccauuacugaguuuag

SEQ ID NO: 94 - RNA, 23 nt, Homo sapiens
uccuguacugagcugccccgagg

SEQ ID NO: 95 - RNA, 20 nt, Homo sapiens
ucguaccgugaguaauaaug

SEQ ID NO: 96 - RNA, 19 nt, Homo sapiens
aauugcacgguauccaucu

SEQ ID NO: 97 - RNA, 21 nt, Homo sapiens
ugagugugugugugugagugu

SEQ ID NO: 98 - RNA, 22 nt, Homo sapiens
ugagguaguagguugugugguu

SEQ ID NO: 99 - RNA, 20 nt, Homo sapiens
uacaguauagaugauguacu

SEQ ID NO: 100 - RNA, 21 nt, Homo sapiens
cacgcucaugcacacacccac

SEQ ID NO: 101 - RNA, 21 nt, Homo sapiens
ugagguaguagguuguguggu

SEQ ID NO: 102 - RNA, 19 nt, Homo sapiens
agcagcauuguacagggcu

SEQ ID NO: 103 - RNA, 21 nt, Homo sapiens
cguaccgugaguaauaaugcg

SEQ ID NO: 104 - RNA, 24 nt, Homo sapiens
gaaaccguuaccauuacugaguuu

SEQ ID NO: 105 - RNA, 21 nt, Homo sapiens
uaaagugcugacagugcagau

SEQ ID NO: 106 - RNA, 22 nt, Homo sapiens
ugagguaguaguuugugcuguu

SEQ ID NO: 107 - RNA, 15 nt, Homo sapiens
uuaccauuacugagu

SEQ ID NO: 108 - RNA, 19 nt, Homo sapiens
aaugacacgaucacucccg

SEQ ID NO: 109 - RNA, 20 nt, Homo sapiens
ccaauauuacugugcugcuu

SEQ ID NO: 110 - RNA, 23 nt, Homo sapiens
ucuacagugcacgugucuccagu

SEQ ID NO: 111 - RNA, 23 nt, Homo sapiens
gaaaccguuaccauuacugaguu

SEQ ID NO: 112 - RNA, 21 nt, Homo sapiens
uaaggugcaucuagugcagau

SEQ ID NO: 113 - RNA, 22 nt, Homo sapiens
ucguaccgugaguaauaaugcg

SEQ ID NO: 114 - RNA, 21 nt, Homo sapiens
ugucuuacucccucaggcaca

SEQ ID NO: 115 - RNA, 22 nt, Homo sapiens
guaguguuuccuacuuuaugga

SEQ ID NO: 116 - RNA, 21 nt, Homo sapiens
guaguguuuccuacuuuaugg

SEQ ID NO: 117 - RNA, 23 nt, Homo sapiens
ugagcgccucgacgacagagccg

SEQ ID NO: 118 - RNA, 22 nt, Homo sapiens
acugcagugaaggcacuuguag

SEQ ID NO: 119 - RNA, 28 nt, Homo sapiens
cccccacugcuaaauuugacuggcuuuu

SEQ ID NO: 120 - RNA, 31 nt, Homo sapiens
gcugguccgaugguaguggguuaucagaacu

SEQ ID NO: 121 - RNA, 23 nt, Homo sapiens
agcuacauugucugcuggguuuc

SEQ ID NO: 122 - RNA, 22 nt, Homo sapiens
auauaauacaaccugcuaagug

SEQ ID NO: 123 - RNA, 23 nt, Homo sapiens
cagugcaauguuaaaagggcauu

SEQ ID NO: 124 - RNA, 22 nt, Homo sapiens
uuauaaagcaaugagacugauu

SEQ ID NO: 125 - RNA, 22 nt, Homo sapiens
acaguagucugcacauugguua

SEQ ID NO: 126 - RNA, 23 nt, Homo sapiens
aucacauugccagggauuuccaa

SEQ ID NO: 127 - RNA, 23 nt, Homo sapiens
ucaagagcaauaacgaaaaaugu

SEQ ID NO: 128 - RNA, 22 nt, Homo sapiens
cagugcaauguuaaaagggcau

SEQ ID NO: 129 - RNA, 21 nt, Homo sapiens
uuaugguuugccugggacuga

SEQ ID NO: 130 - RNA, 26 nt, Homo sapiens
cccccacugcuaaauuugacuggcuu

SEQ ID NO: 131 - RNA, 22 nt, Homo sapiens
uucaaguaauccaggauaggcu

SEQ ID NO: 132 - RNA, 32 nt, Homo sapiens
ggcugguccgaugguaguggguuaucagaacu

SEQ ID NO: 133 - RNA, 22 nt, Homo sapiens
aucacauugccagggauuucca

SEQ ID NO: 134 - RNA, 22 nt, Homo sapiens
ugagaacugaauuccauggguu

SEQ ID NO: 135 - RNA, 23 nt, Homo sapiens
caacggaaucccaaaagcagcug

SEQ ID NO: 136 - RNA, 31 nt, Homo sapiens
ggcugguccgaugguaguggguuaucagaac

SEQ ID NO: 137 - RNA, 22 nt, Homo sapiens
uauugcacuugucccggccugu

SEQ ID NO: 138 - RNA, 20 nt, Homo sapiens
ugagguaguagguugugugg

SEQ ID NO: 139 - RNA, 21 nt, Homo sapiens
aaaccguuaccauuacugagu

SEQ ID NO: 140 - RNA, 23 nt, Homo sapiens
guaaacauccuugacuggaagcu

SEQ ID NO: 141 - RNA, 21 nt, Homo sapiens
ugagguaguaguuuguacagu

SEQ ID NO: 142 - RNA, 22 nt, Homo sapiens
uccuguacugagcugccccgag

SEQ ID NO: 143 - RNA, 20 nt, Homo sapiens
uagcagcacguaaauauugg

SEQ ID NO: 144 - RNA, 20 nt, Homo sapiens
aaaccguuaccauuacugag

SEQ ID NO: 145 - RNA, 21 nt, Homo sapiens
uggagagaaaggcaguuccug

SEQ ID NO: 146 - RNA, 20 nt, Homo sapiens
ugagguaguagguuguauag

SEQ ID NO: 147 - RNA, 21 nt, Homo sapiens
uauugcacuugucccggccug

SEQ ID NO: 148 - RNA, 21 nt, Homo sapiens
cauugcacuugucucggucug

SEQ ID NO: 149 - RNA, 21 nt, Homo sapiens
accaauauuacugugcugcuu

SEQ ID NO: 150 - RNA, 20 nt, Homo sapiens
ugagguaguagauuguauag

SEQ ID NO: 151 - RNA, 20 nt, Homo sapiens
cauugcacuugucucggucu

SEQ ID NO: 152 - RNA, 21 nt, Homo sapiens
aaugacacgaucacucccguu

SEQ ID NO: 153 - RNA, 21 nt, Homo sapiens
ugaggggcagagagcgagacu

SEQ ID NO: 154 - RNA, 21 nt, Homo sapiens
ucaggcucaguccccucccga

SEQ ID NO: 155 - RNA, 21 nt, Homo sapiens
uccuguacugagcugccccga

SEQ ID NO: 156 - RNA, 20 nt, Homo sapiens
uccuguacugagcugccccg

SEQ ID NO: 157 - RNA, 21 nt, Homo sapiens
ugagguaguaguuugugcugu

SEQ ID NO: 158 - RNA, 20 nt, Homo sapiens
agagguaguagguugcauag

SEQ ID NO: 159 - RNA, 17 nt, Homo sapiens
uccuguacugagcugcc

SEQ ID NO: 160 - RNA, 20 nt, Homo sapiens
ugagguaguaguuugugcug

SEQ ID NO: 161 - RNA, 20 nt, Homo sapiens
ucaggcucaguccccucccg

SEQ ID NO: 162 - RNA, 15 nt, Homo sapiens
ucauguaugaugcug